(19) United States
(12) Patent Application Publication
(10) Pub. No.: US 2025/0268598 A1
(43) Pub. Date: Aug. 28, 2025

(54) TRAUMA BELT FOR NON-TOURNIQUETABLE HEMORRHAGE CONTROL

(71) Applicants: Nina Mclain, Philadelphia, MS (US); Andrew P. Dabbs, Philadelphia, MS (US); Derek Patton, Hattiesburg, MS (US)

(72) Inventors: Nina Mclain, Philadelphia, MS (US); Andrew P. Dabbs, Philadelphia, MS (US); Derek Patton, Hattiesburg, MS (US)

(73) Assignee: The University of Southern Mississippi, Hattiesburg, MS (US)

(21) Appl. No.: 19/061,731

(22) Filed: Feb. 24, 2025

Related U.S. Application Data
(60) Provisional application No. 63/557,219, filed on Feb. 23, 2024.

(51) Int. Cl. A61B 17/12; A61B 17/00

(52) U.S. Cl. CPC A61B 17/12009; A61B 2017/00477; A61B 2017/00526; A61B 2017/00862; A61B 2017/00889; A61B 2017/00893; A61B 2017/12004

(57) ABSTRACT

A trauma belt (1) for non-tourniquetable hemorrhage control of a patient having an entry wound in a shoulder bundle, groin, femoral, abdominal, back, or chest region, the trauma belt (1) includes a rigid top arm (70), a rigid bottom arm (80) and a stem (40) connecting the top arm (70) and the bottom arm (80), a pad (100) connected to the interior surface (20) of the top arm (70), wherein the stem (40) is configured to provide constricting force between the top arm (70) and bottom arm (80) to provide pressure of the pad (100) against the entry wound.

### BACKGROUND

Warfighter battlefield wounds to the highly vascular shoulder bundle, groin/femoral, abdominal, back, and chest regions are not compressible using tourniquets. These vascular-rich areas, when injured, cause rapid exsanguination if immediate and consistent pressure is not applied. Immediate application of pressure is key to hemorrhage control.¹ Current technologies fall short of providing desired attributes for the injured warrior, especially under battlefield/warfare conditions.

The number one preventable cause of death in the military is due to battlefield trauma hemorrhage in the pre-hospital setting.^{2,3} In a 2020 study of Special Operations Command battlefield fatalities, 88% of potentially survivable injuries included hemorrhage as a factor in the mechanism of death while 25% of deaths on the battlefield are preventable.^{4,5} In the civilian population trauma hemorrhage is cited as the second leading cause of death.⁶ While many advances have been made in the area of antifibrinolytics and hemostatic dressings, availability, supply chain disruptions, and untoward effects can leave our warfighters in critical states.

Tourniquet practices have long been the mainstay of controlling exsanguination for military and civilian populations, although success rates for this practice are suboptimal in junctional and torso regions.^{7,8} Junctional sites include the shoulder bundle, groin, and neck regions. Undoubtably, tourniquets increase survivability in extremity trauma, yet they are not without complications and limitations. Motor and sensory nerve palsy and damage, as well as tissue and reperfusion injuries can occur with the use of extremity tourniquets, and these may contribute to prolonged rehabilitation or permanent disabilities.⁹

The basic need to control junctional or torso hemorrhage in the war zone setting, immediately and effectively, using constant pressure remains the optimal treatment for hemorrhagic control.¹⁰ While further studies are needed, junctional tourniquets may be useful in certain situations, but they are not a replacement for direct pressure or invasive interventions.⁹ Injuries to the torso and junctional areas such as the groin and shoulder are treated in the field with a “pack and wrap” approach as taught in the advanced trauma life support (ATLS) course.¹¹ Under battlefield conditions and during subsequent medical evacuation, packing and wrapping the wound does not supply firm, direct pressure, and is easily dislodged during movement.

Military trauma care training for warfighters from the Tactical Combat Casualty Care (TCCC) guidelines includes wound packing, torniquet use, and hemostatic gauze.¹¹ Long-term effects of hemostatic gauze have not been fully explored as they are a relatively recent development, although some studies have shown exothermic cellular damage to tissues.^{12,13} Hemostatic dressings and substances are still evolving but currently, none provide ideal hemorrhage control without potential negative effects and some still require tourniquet use in addition to their application.¹⁴

Medical resource supply in far forward facilities and locations presents concerns when mass casualty or prolonged warfare occurs.¹⁵ A 2022 Delphi study concluded that bandages, junctional and limb tourniquets, pelvic binders/stabilizers and hemostatic agents should be included in the standard military equipment.¹⁶

The REBOA product introduced in the early 2000s is an invasive device inserted through the femoral artery and inflated in the abdominal aorta to reduce or eliminate distal hemorrhage.¹⁷ To insert a REBOA, extensive training and expertise is required and, as a result, the REBOA is usually placed by a vascular surgeon, interventional radiologist, or other specially trained physician. This, along with the requirement to insert the REBOA under sterile conditions makes the product unusable under battlefield conditions as the first responder is frequently a fellow warfighter and the conditions are far from sterile.

The CROC Clamp was designed as a tourniquet for the junctional inguinal area.¹⁸ The clamp consists of five metal pieces and two straps that are provided in unassembled form. A multi-piece device that requires assembly is not acceptable during real-time “under fire” conditions. Another major disadvantage to the CROC Clamp is that it can be easily dislodged during evacuation due to rotational forces.

Another junctional tourniquet developed for military use is the SAM Junctional Tourniquet. The SAM Junctional Tourniquet is a rigid belt with an inflatable bladder that is positioned over the injury. While evidence demonstrates that the SAM Junctional Tourniquet is applied more rapidly than the CROC Clamp during simulated evacuations, it does not offer compression of the wound between two rigid “sandwiched” plates with flexible portions to allow expansion during breathing.¹⁹

With the current therapies failing to meet the desired standards for battlefield conditions, a need exists for a device that is:
Rapidly and easily applied by one person.
Reliable and secure in the face of rotational forces of movement and med-evac.
Ultra-lightweight, portable.
Provides consistent, required pressure to stop or significantly slow hemorrhage.
Functional for torso and junctional wounds.
Usable in all environments of battle and climate.
A single unit, no assembly required.
3D printable onsite or via naval vessel or FOB for resupply.
Cost-effective and also applicable for civilian casualty care.

The primary goal of hemorrhage control is to immediately apply constant pressure and subsequently move or evacuate the injured to safety. By doing this, operational effectiveness and casualty outcomes will be significantly improved.

### SUMMARY OF THE INVENTION

The present invention relates to a trauma belt (T-Belt) that can provide hemorrhage control in the battlefield and under fire settings to allow a wounded solider to be dragged or evacuated to safety without dislodgement of the device and to provide superior performance when compared to the pack and wrap approach.
1. In a first aspect, the present invention relates to a trauma belt (1) for non-tourniquetable hemorrhage control of a patient having an entry wound in a shoulder bundle, groin, femoral, abdominal, back, or chest region, said trauma belt (1) comprising a rigid top arm (70), a rigid bottom arm (80) and a stem (40) connecting the top arm (70) and the bottom arm (80),
   a pad (100) connected to an interior surface (20) of the top arm (70),
   wherein the stem (40) is configured to provide a constricting force between the top arm (70) and bottom arm (80) to provide pressure of the pad (100) against the entry wound.
2. The trauma belt (1) of sentence 1, further comprising a second pad (100) connected to an interior surface (50) of the bottom arm (80) and positioned to press against an exit wound.
3. The trauma belt (1) according to any one of sentences 1-2, wherein the pad (100) is detachable from the trauma belt (1).
4. The trauma belt (1) according to any one of sentences 1-3, wherein the pad (100) has a compression plate (110) and a pressure pad (120).
5. The trauma belt (1) according to any one of sentences 1-4, wherein the pressure pad (120) has a shape selected from block type (120a), curved edges (120b), trapezoidal shaped (120c), and hemispherical shaped (120d).
6. The trauma belt (1) according to any one of sentences 1-5, wherein the compression plate (110) is rigid or semi-rigid.
7. The trauma belt (1) according to any one of sentences 1-6, wherein the stem (40) comprises at least one joint adapted to allow folding of the trauma belt (1) for storage.
8. The trauma belt (1) according to any one of sentences 1-7, wherein the trauma belt (1) is formed using additive manufacturing.

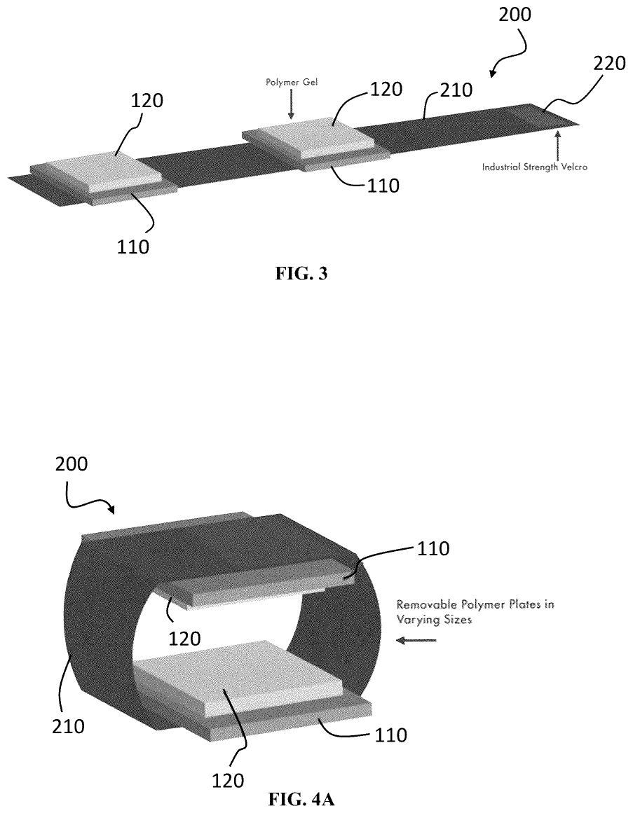

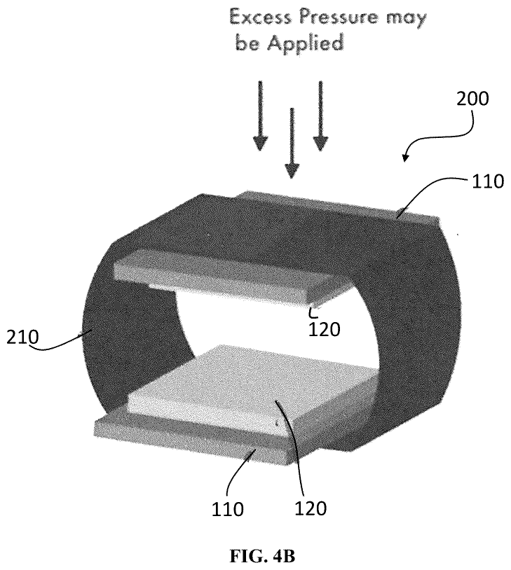

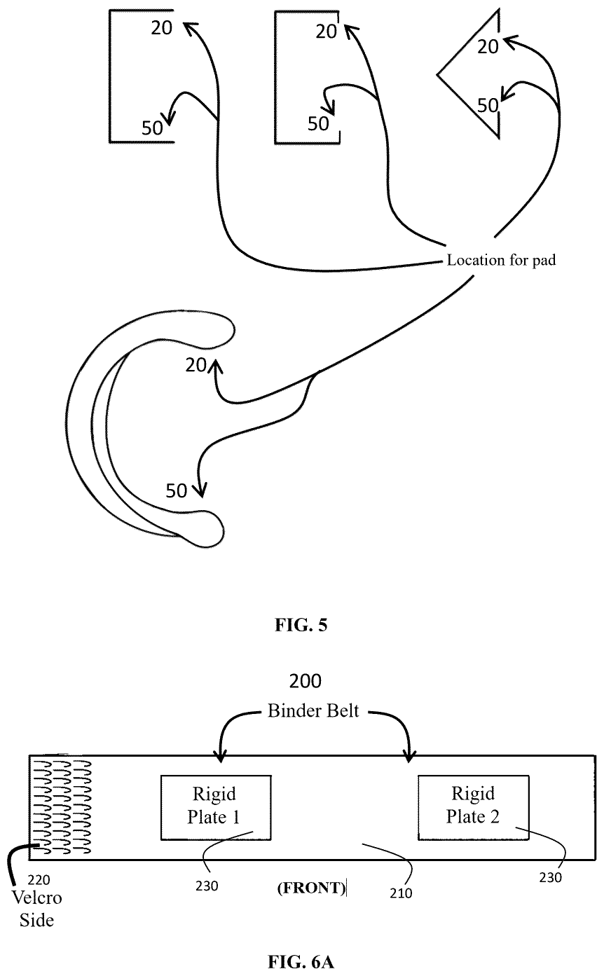

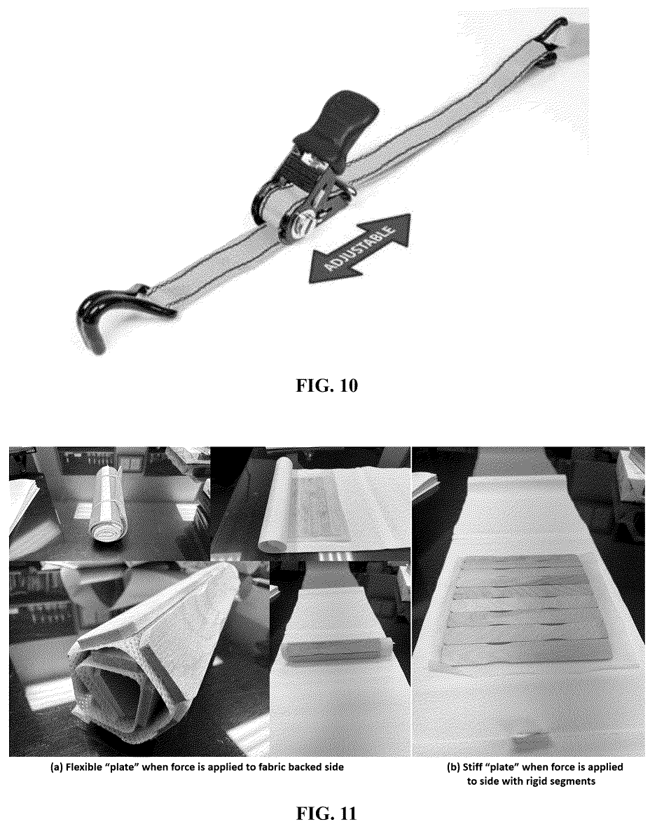

19. The trauma belt (1) of sentence 8, wherein an exterior wall of the trauma belt (1) has a thickness of from about 10 mm to about 30 mm, or from about 15 mm to about 25 mm.
10. The trauma belt (1) of any one of sentences 8-9, wherein the trauma belt (1) has an internal infill percentage of from about 10% to about 100%,
   or from about 10% to 50%, or from about 15% to 30%, based on the total volume of the trauma belt (1), such that the pad (100) provides sufficient tension to achieve a pressure of 30-60 mm Hg, when the trauma belt is applied to the shoulder bundle, the back, or the chest,
   or from about 50% to about 90%, or from about 60% to about 80, based on the total volume of the trauma belt (1) such that the pad (100) provides sufficient tension to achieve a pressure of 60 mm Hg-200 mm Hg, when the trauma belt is applied to the femoral, the back, or the chest,
   or from about 90% to 100%, or from about 95% to 100%, or about 100%, based on the total volume of the trauma belt (1) such that the pad (100) provides sufficient tension to achieve a pressure of 500 mm Hg-650 mm Hg, when the trauma belt is applied to the abdomen, or the groin.
11. The trauma belt (1) according to any one of sentences 1-10, wherein the infill further comprises a fiber reinforcement selected from the group consisting of nylon and carbon fiber.
12. The trauma belt (1) according to any one of sentences 1-11, wherein a coupler (220) connects an end of the top arm (70) to an end of the bottom arm (80) to reduce rotational movement of the trauma (1) belt during use.
13. The trauma belt (1) of sentence 1, wherein the coupler (220) contains a hook-and-loop fastener or a buckle.
14. The trauma belt (1) according to any one of sentences 12-13, wherein the coupler (220) is detachable from the trauma belt (1).
15. The trauma belt (1) according to any one of sentences 1-14, wherein the trauma belt (1) has a general configuration as depicted in one of the embodiments of FIGS. 5, 8, and 12B.
16. The trauma belt (1) according to any one of sentences 1-15, wherein the trauma belt (1) is configured to have sufficient tension to ensure that the pad (100) provides 20-700 mm Hg pressure inwardly against the wound, or from about 30-50 mg Hg pressure inwardly against the wound, or
   when applied to the abdomen, provides 615-635 mm Hg pressure inwardly against the wound, or
   when applied to the groin provides about 505-544 mm Hg pressure inwardly against the wound, or
   when applied to the shoulder bundle provides about 30-60 mm Hg pressure inwardly against the wound, or
   when applied to the femoral provides about 60-100 mm Hg pressure inwardly against the wound,
   when applied to the back provides about 30-100 mm Hg pressure inwardly against the wound,
   when applied to the chest provides about 30-100 mm Hg pressure inwardly against the wound.
17. The trauma belt (1) according to any one of sentences 1-16, wherein the thickness of the compression plate (110) is from 0.1 inches to 1 inch, or 0.2 inches to 0.8 inches, or about 0.25 inches.
18. The trauma belt (1) according to any one of sentences 1-17, wherein the area of the pad (100) in the horizontal plane is 4 square inches to 36 square inches, or 10 square inches to 32 inches, or about 16 square inches.
19. The trauma belt (1) according to any one of sentences 1-18, wherein the compression plate (110) is sufficiently pliable to be able to conform to the curvature of the body part being bandaged with hand manipulation and also able to retain a conformed shape.
20. The trauma belt (1) according to any one of sentences 1-19, wherein the pad (100) is detachable from the trauma belt (1).
21. The trauma belt (1) according to sentence 20, wherein the pad (100) is detachably attached to the trauma belt (1) by a loop or a hook-and-loop fastener.
22. The trauma belt (1) according to any one of sentences 1-21, wherein the pad (100) further comprises one or more components selected from the group consisting of a quick clot, an anticoagulant, an anesthetic, and an antimicrobial.
23. The trauma belt (1) according to any one of sentences 1-22, wherein a distance between a lower surface of the pad (100) attached to the top arm (70) and an upper surface of the bottom arm (80) is from about 6 inches to about 24 inches, or from about 8 inches to about 15 inches or about 12 inches.
24. The trauma belt (1) according to any one of sentences 1-23, wherein a distance between a lower surface of the pad (100) attached to the top arm (70) and an upper surface of the pad (100) attached to the bottom arm (80) is from about 6 inches to about 24 inches, or from about 8 inches to about 15 inches, or about 12 inches.
25. In a second aspect, the present invention relates to a stretchable trauma belt (200) for non-tourniquetable hemorrhage control of a patient having an entry wound in a shoulder bundle, groin, femoral, abdominal, back, or chest region, said trauma belt (200) including:
   a stretchable belt (210),
   a pad (230) adhered to the stretchable belt (210),
   a fastener (220) configured to fasten a first end and a second end of the stretchable trauma belt (200) to form a closed loop as shown in FIGS. 4, 6 and 7 to provide a constricting force and a pressure of the pad (230) against the entry wound.
26. The stretchable trauma belt (200) of sentence 25, comprising a plurality of pads (230).
27. The stretchable trauma belt (200) of any one of sentences 25-26, wherein the one or more pad(s) (230) are detachable from the stretchable belt (210).
28. The stretchable trauma belt (200) according to any one of sentences 25-27, wherein the pad (230) comprises a pressure pad (250) and a compression plate (240) having a top side adhered to the pressure pad (250) and a bottom side adhered to the stretchable belt (210).
29. The stretchable trauma belt (200) according to any one of sentences 25-28, wherein the pressure pad (250) has a shape selected from block type (250a), curved edges (250b), trapezoidal shaped (250c), and hemispherical shaped (250d).
30. The stretchable trauma belt (200) of according to any one of sentences 25-29, wherein the pad comprises a compression plate (240) that is rigid or semi-rigid.
31. The stretchable trauma belt (200) according to any one of sentences 25-30, wherein a distance between inward facing surfaces of each of the two or more pads (230) is from about 6 inches to about 24 inches, or from about 8 inches to about 24 inches, or about 12 inches.
32. The stretchable trauma belt (200) of sentence 25, wherein each of the plurality of pads (230) are arranged side by side along the stretchable belt (210) such that their longitudinal edges are adjacent to each other.
33. The stretchable trauma belt (200) according to any one of sentences 25-30, wherein a distance between an inward facing surface of the pad (230) and an inner surface of the stretchable belt (210) at a point opposite the pad (230) in the closed loop is from about 6 inches to about 24 inches, or from about 8 inches to about 15 inches, or about 12 inches.
34. In a third aspect, the present invention relates to a trauma belt for hemorrhage control of a patient having an entry wound in a shoulder bundle, groin, femoral, abdominal, back, or chest region, said trauma belt comprising a stretchable band that is 2 inches to 16 inches wide comprising fasteners
   wherein two rigid pads are connected to the stretchable band, and said trauma belt is configured so that, in use, one said rigid pad is positioned over an entry wound and the other said rigid pad is positioned over an exit wound, and said trauma belt provides a constricting force between the two pads so as to apply pressure of the pads against the respective entry and exit wounds as shown in FIGS. 3, 4A, 4B, 6A-6D and 7, and
   the pads optionally have at least component selected from the group consisting of: a) clotting materials suitable for hemostasis; b) medications such as antibiotics to ward off infection; and c) a polymer which hardens when in contact with proteins in blood.
35. The trauma belt according to sentence 34, wherein the trauma belt is configured to recoil when opened.
36. The trauma belt according to any one of sentences 1-21 and 34-35 and the stretchable trauma belt of any one of sentences 22-33, wherein the fasteners are hook-and-loop fasteners, a first end of the belt comprises hook fasteners and a second end of the belt comprises loop fasteners as shown in FIGS. 3, 4A, 4B, 6A-6D, and 7.
37. The trauma belt according to any one of sentences 1-21 and 34-35 and the stretchable trauma belt of any one of sentences 22-33, wherein the fasteners comprise double ring fasteners, a first end of the belt comprises two rings and a second end of the belt is configured to loop through the two rings to thereby fasten the first and second ends together.
38. The trauma belt according to sentence 37, wherein the first and second ends of the stretchable trauma belt are fastened together by pulling the second end of the belt through both rings of the first end of the belt, separating the two rings so that there is enough space for the second end of the belt to pass between them, looping the second end of the belt back towards the two rings so that the second end of the belt folds over itself, pulling the second end of the belt through the rings by going over the closest ring and under the second ring, and pulling the loose second end of the belt until the belt tightens sufficiently. Exemplary double ring fasteners are shown in FIG. 9 as used in connection with a belt for pants.
39. The trauma belt according to sentence 34, wherein the fasteners comprise a first loop at a first end of the belt and a second loop at a second end of the belt which loops are cinched together with a fastener belt comprising a first hook on a first end of the fastener belt and a second hook on a second end of the fastener belt, wherein the first hook is hooked through the first loop and the second hook is hooked through the second loop, and the fastener belt optionally has a sliding ratchet for shortening the fastener belt, as exemplified in FIG. 10.

### DETAILED DESCRIPTION OF THE DRAWINGS

FIG. 1 shows a schematic of the trauma belt (1) according to one embodiment of the present invention, without the pad (100).

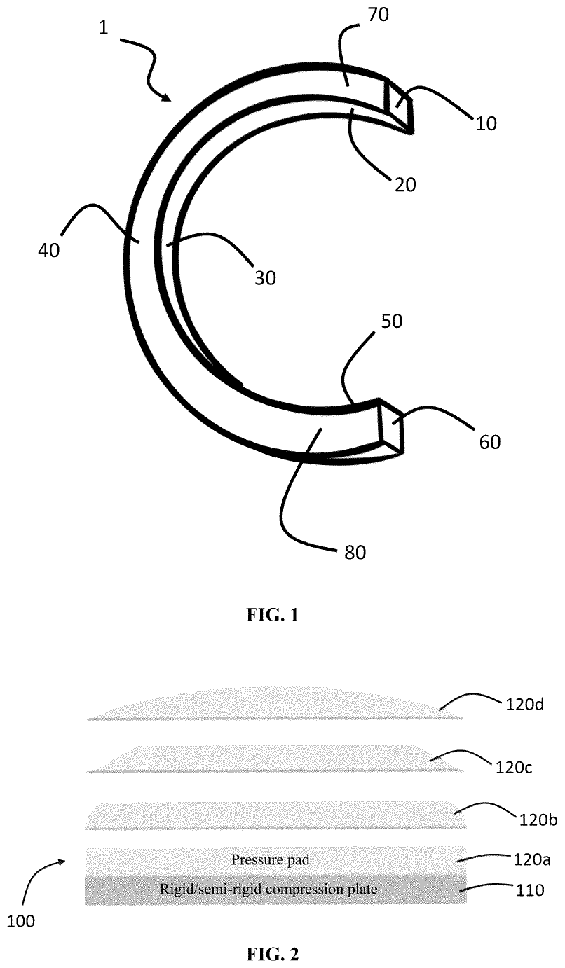

FIG. 2 shows four possible cross sectional shape profiles (120a/120b/120c/120d) of the pressure pad (120) in combination with the rigid/semi-rigid compression plate (110).

FIG. 3 shows a perspective view of a schematic of the trauma belt (1) including two pressure pads (120) and corresponding rigid plates (110), where the pressure pads (120) are made of a polymer gel. The depicted trauma belt (1) also includes an industrial strength hook-and-loop fastener (220) and a stretchable belt (210).

FIG. 4A and FIG. 4B show perspective views of the schematics of the trauma belt (1) as shown in FIG. 3, when the edges of the belt are connected together, using the hook-and-loop fastener (220) to form a loop.

FIG. 5 shows a schematic of the trauma belt (1) according to a variety of different embodiments according to the present invention.

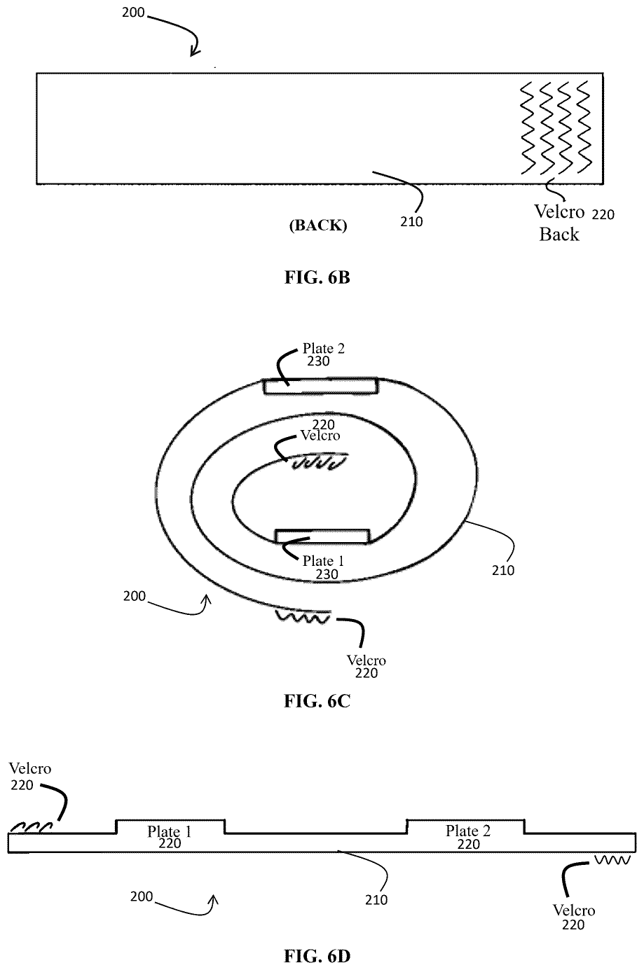

FIG. 6A shows a front view of a schematic of the trauma belt (1) according to the present invention.

FIG. 6B shows a back view of a schematic of the trauma belt (1) shown in FIG. 6A.

FIG. 6C shows a top side view of a schematic of the trauma belt (1) shown in FIGS. 6A-6B.

FIG. 6D shows a side view of a schematic of the trauma belt (1) shown in FIGS. 6A-6C.

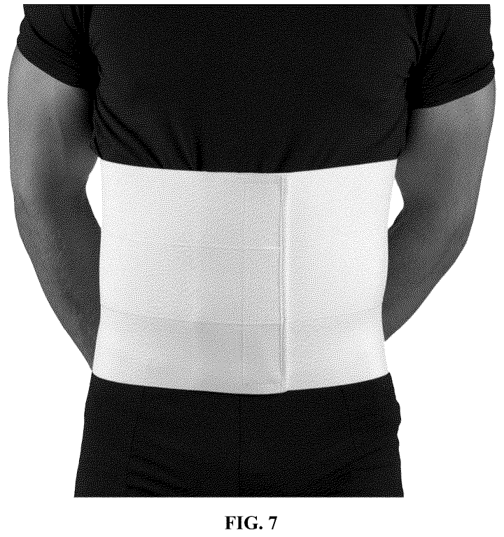

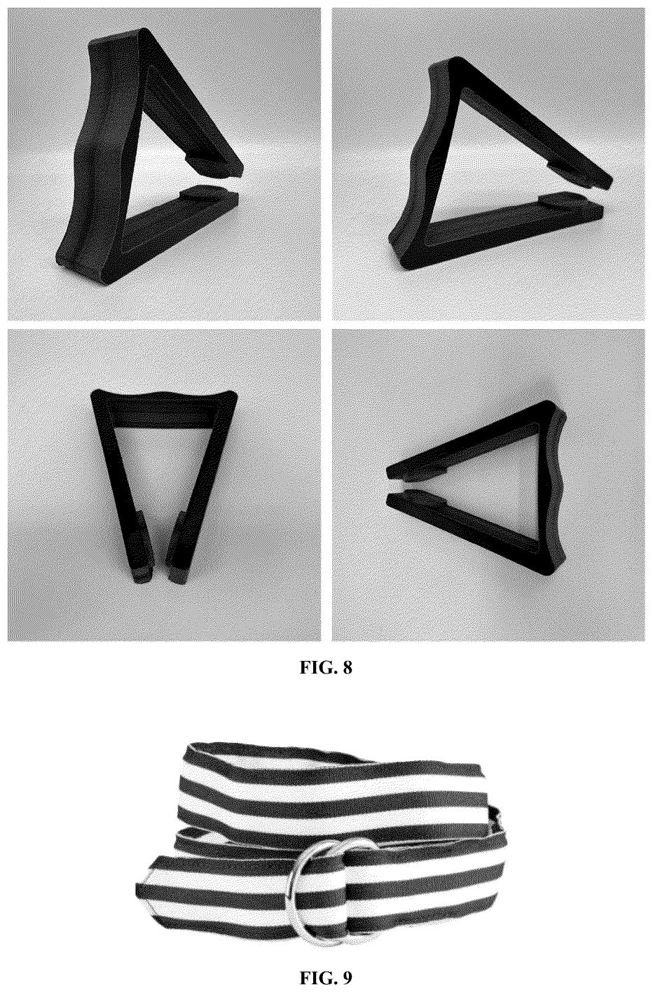

FIG. 7 shows a photograph of an embodiment of the trauma belt (1) without pads.

FIG. 8 shows photographs of a 3D printed trauma belt (1) according to the present invention. The trauma belt of FIG. 8 was printed with a fused deposition molding printer using nylon resin. The 3D printed trauma belt (1) is capable of applying approximately 40 N of force when the arms are flexed to a parallel position.

FIG. 9 shows a photograph of a double ring fastener.

FIG. 10 shows a fastener belt that includes a sliding ratchet.

FIG. 11 shows a photograph of a trauma belt according to an embodiment of the present invention that includes a directionally flexible structure that exhibits asymmetric stiffness by integrating a series of rigid segments adhered to a flexible substrate.

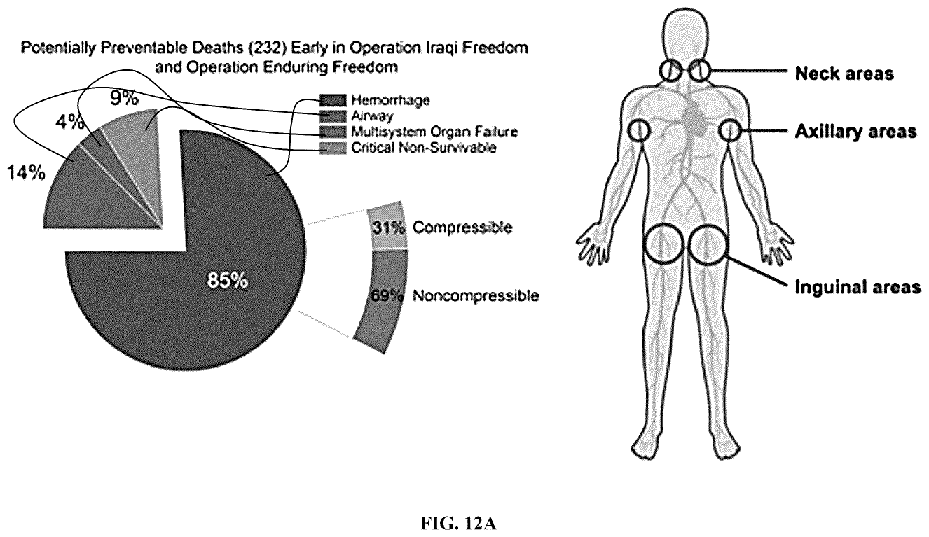

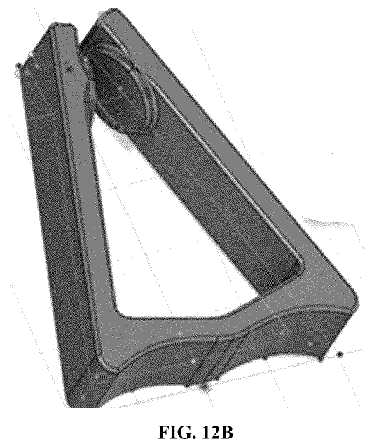

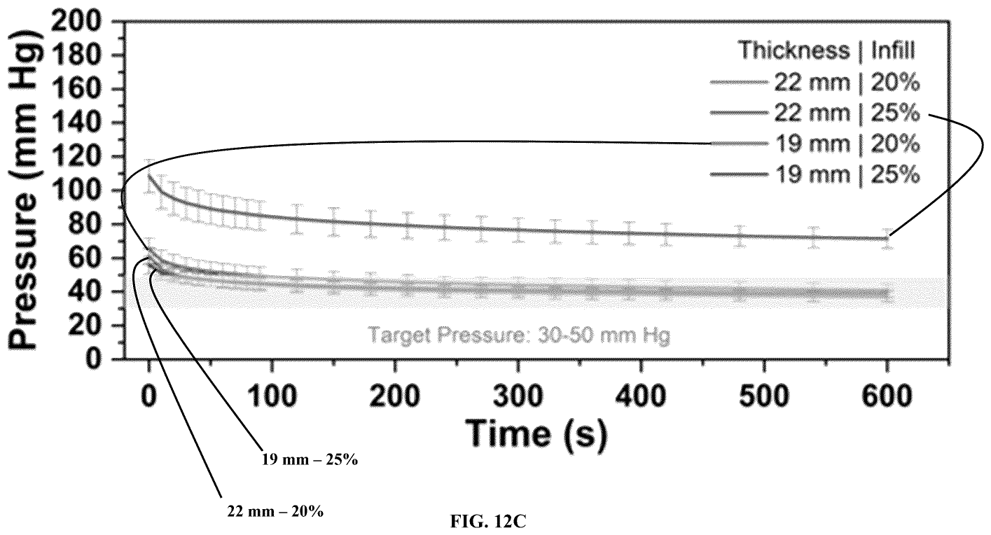

FIG. 12A shows a chart of the potentially preventable deaths during combat, based on 232 deaths.

FIG. 12B shows a computer-generated schematic view of a 3D printed trauma belt as shown in FIG. 8.

FIG. 12C shows a chart demonstrating the ability of a variety of thicknesses and infill percentages utilized in the 3D printed trauma belt to achieve pressures suitable to control hemorrhaging.

### DETAILED DESCRIPTION OF INVENTION

The present invention relates to a T-Belt that can provide hemorrhage control on the battlefield and in under fire settings to allow a wounded soldier to be dragged or evacuated to safety without dislodgement of the T-Belt and with superior results when compared to the often used pack and wrap approach.

The trauma belt (1) of the present invention is for non-tourniquetable hemorrhage control of a patient having an entry wound in a shoulder bundle, groin, femoral, abdominal, back, or chest region. The trauma belt (1) may include:
a rigid top arm (70),
a rigid bottom arm (80);
a stem (40) connecting the top arm (70) and the bottom arm (80), and
a pad (100) connected to an interior surface (20) of the top arm (70), and
wherein the stem (40) is configured to provide constricting force between the top arm (70) and bottom arm (80) to cause the pad (100) to exert pressure against the entry wound.

Suitable methods for forming the trauma belt of the present invention include additive manufacturing, for example, material extrusion, binder jetting, powder bed fusion, directed energy deposition, 3D printing and material jetting. The trauma belt may include a solid exterior wall and an internal infill pattern The solid exterior wall may have a thickness of from about 10 mm to about 30 mm, or from about 15 mm to about 25 mm. The trauma belt may have an infill percentage of from 10-100%. Preferably, the trauma belt (1) comprises an internal infill percentage of from about 10% to about 30%, or from about 15% to 30%, or from about 20% to about 25%, based on the total volume of the trauma belt. The exterior wall thickness and infill percentage based on volume, can be adjusted such that the desired target pressure on the wound can be achieved.

Alternatively, the size of the pressure pad can be changed to alter the amount of pressure applied to the wound. This can be achieved by supplying the device with multiple interchangeable contact pads of different sizes. In a field/emergency setting, the contact pad could be any object placed under surface 20 of the device in contact with the location where pressure is required. A larger pressure pad would apply less pressure since the force is spread over a larger area, than a smaller pressure pad which spreads the same force over a smaller area.

Another embodiment of the invention is a stretchable trauma belt (200) for non-tourniquetable hemorrhage control of a patient having an entry wound in a shoulder bundle, groin, femoral, abdominal, back, or chest region. The stretchable trauma belt (200) includes:
a stretchable belt (210),
a pad (230) adhered to the stretchable belt (210),
a fastener (220) configured to fasten first and second ends of the stretchable trauma belt (200) to form a closed loop as shown in FIGS. 4, 6, 7 and 12 to provide a constricting force to cause exertion of pressure of the pad (230) against the entry wound.

The stretchable trauma belt (200) may be a directionally flexible structure that exhibits asymmetric stiffness that is provided by integrating a series of rigid segments in the belt (200), also referred to herein as compression pads, or rigid plates, adhered to a flexible substrate such as a stretchable belt. This stretchable trauma belt (200) provides mechanical support and rigidity when compressed in one direction while permitting flexibility and rollability when compressed in the opposite direction.

In this embodiment, the stretchable trauma belt (200) comprises a plurality of pads, or elongate rigid plates, for example, wooden or polymeric slats, arranged in parallel along their longitudinal axes, and affixed to a continuous, compliant backing layer, for example, a high-strength textile, elastomeric sheet, or polymer film. The rigid elements are positioned such that their longitudinal edges are adjacent or in close contact, allowing for load transfer between elements when the structure is compressed from the rigid side. This results in a substantially rigid plate-like configuration capable of bearing weight and resisting deformation.

Conversely, when force is applied from the opposite side, the backing layer, or stretchable belt material, is placed in tension, and the rigid elements are free to pivot or separate slightly, enabling the structure to roll, fold, or flex in a controlled manner. This asymmetry of the mechanical response allows the structure to function as a deployable or adaptive surface that is stiff under certain loading conditions but is also flexible when deformation is desired.

The T-belt is rugged, durable, lightweight, and, optionally, camo-colored. The T-belt is suitable for transportation in a backpack and can be applied by a single person without the need for extensive training or qualifications.

The T-belt comprises a singular belt system and may include slightly flexible lateral portions and two rigid polymer components. See, for example, FIGS. 3 and 4. When positioned over a wound, pressure is applied to the affected area without compromising ventilation or blood flow to other areas. Unlike current devices on the market, stability is not a factor when rotational forces are present, such as when a wounded soldier startles awake, is combative due to confusion, is being dragged to safety, or is being evacuated under fire.

The T-belt does not require assembly as it can be prepackaged ready for application, saving time in use to allow rapid deployment to control hemorrhaging. The design allows for application to the shoulder, groin, back, chest, and abdominal regions, areas where torniquets are unusable. The T-belt can be packaged in a roll, like an Ace™ bandage. The “rapid on” design makes it easy to use. Once in place, it is secure and stable. The T-belt can be used in any environment whether mountainous terrain, snow and ice, desert, undersea, jungle, aboard ships, in space, a MASH unit, aircraft, hospital, or in an ambulance.

Advantages of the present invention include:
a) Utilization of the T-Belt provides superior control over torso and junctional area hemorrhage, when compared to existing clamp and tourniquet devices;
b) Utilization of the T-Belt provides superior control over torso and junctional region hemorrhage, when compared to the traditional pack and wrap approach; and
c) Utilization of the T-Belt can maintain improved control over torso and junctional region hemorrhage during casualty movement, when compared to current technologies and practices.

### Significance

Exsanguination and death due to hemorrhage remains the leading cause of death on the battlefield. Although tourniquet use and operational strategy changes have improved outcomes for such trauma victims, even one preventable death is considered unacceptable. Warfighters and service persons devoting their lives to protect our country, our liberties, and those of our allies deserve not only the best possible care and equipment available but also deserve a research community that refuses to accept substandard equipment and processes. Currently, the products available to injured warfighters are considered unacceptable as such products do not provide optimal hemorrhage control. The critical barrier to progress with the use of tourniquets is that tourniquets cannot be applied to the abdomen, anterior and posterior chest, groin, shoulder, and neck regions. Other devices that attempt to meet this need provide some benefits under certain conditions such as in a hospital environment, a non-transport situation, or for extremely short periods of time. However, on the battlefield or in under fire conditions these alternative devices fall short of optimal performance or are not feasible for use under these conditions. For example, antifibrinolytics, hemostatic dressings and starches have been used with some success but not without some negative effects. This invention addresses the importance of improving hemorrhage control and the barriers presented by on battlefield trauma care.

A method to stop hemorrhage that is easily and rapidly applied under battlefield conditions and can simulate the first-line approach of manually applied pressure is desirable. The use of the T-belt can provide hands-free treatment of the hemorrhage for the rescuer. The T-belt improves technical capabilities and trauma care due to its rapid deployment and positioning over the hemorrhagic wound, onsite. Furthermore, the T-belt remains secure in position and can maintain pressure on the wound even through the rotational forces of movement and evacuation of the subject, a problem with current clamp technologies. Other belt devices do not allow for expansion of the lungs and thorax due to their encompassing rigid nature whereas the T-belt has flexible side panels to permit expansion of the lungs and thorax. This provides a method to control hemorrhage even when rotational forces are present, thereby providing care superior to that currently delivered to our warfighters. In this manner, the T-belt can aid in maintaining oxygenation of tissues already in a precarious state by not restricting respiration as may the case with current options. Further, exothermic tissue reactions, surgical difficulties and complications and exposure issues related to use of copious amounts of artificial clotting substances once at a surgical facility, and prevention of associated pneumonias from restricted thorax expansion can be reduced or prevented.

The T-belt technology eliminates the need for devices that must be assembled before use, expensive products that often cause surgical difficulties and, in many cases long-term tissue damage post-evacuation

### Innovation

The invention seeks to shift battlefield hemorrhage control from current approaches such as the application of clotting substances or cumbersome clamp devices to an easily applied simple device. By demonstrating effectiveness in reducing blood loss, the T-belt shifts the focus to preventing injuries.

The T-Belt has advantages over existing products. The CROC clamp requires assembly prior to application and is subject to dislodgement by the rotational forces created by casualty transport and movement. The T-Belt can remain stable and secure even when subjected to such rotational forces.

The SAM Junctional Tourniquet (SAM JT) has three distinct disadvantages, weight, deflation potential, and restriction of respiration. Due to the weight advantage alone, there will be a preference by the Warfighter for the T-Belt weighing one pound over the three-pound SAM JT. Further, an inflatable bladder component of the SAM JT makes it susceptible to puncture or deflation whereas the T-Belt is a singular piece. The non-flexible belt of the SAM JT restricts the normal chest and abdominal movements of breathing. The T-Belt can have two panels with flexibility built in to allow for normal chest and abdominal movements of breathing thereby reducing or preventing post injury pneumonia sometimes caused by restricted lung inflation. Antifibrinolytics and hemostatic dressings are effective but are not without their problems. Post-evacuation surgical removal of clotting products is tedious and exothermic tissue damage produces necrotic tissue and can delay healing.²⁰ The T-Belt does not produce heat.

Finally, the REBOA is not able to be used outside of sterile environments and must be placed by a surgeon or other specially trained physician. The T-Belt can be applied by a medic or fellow soldier.

### T-Belt Detail

The T-Belt can be a lightweight, portable belt-clamp device made of lightweight strong polymer materials with an adjustable, secured hook-and-loop fastener strapping system. The “belt” portion can be 8″ wide×40″ long with bilayers composed of rigid polymer compression plates and pressure pads positioned anteriorly and posteriorly. The semi-elastic polymer belt can be secured with one bilayer plate directly over the wound. The device can be tightened and secured with the heavy-duty hook-and-loop fastener. The rigid polymer plates allow compression in an anterior to posterior direction, and are not affected by rotational forces, movement, or evacuation of the subject even under extreme battlefield circumstances.

The differentiation from the prior devices is that the T-belt is designed for external abdominal, shoulder girdle, groin, or femoral pressure where tourniquets cannot be applied and where high volume hemorrhage occurs when these areas are injured.

### Effect of Shape and Materials Composition of Polymeric Compression Plates/Pads on The Contact Pressure Profile

An example of the T-belt is illustrated in FIGS. 3 and 4. The “belt” portion may be fabricated using commercially available medical-grade elastic webbing equipped and/or modified with an industrial strength hook-and-loop fastener Multiple modes of plastics processing can be applied, including traditional processes such as injection molding and more contemporary processes such as additive manufacturing (e.g., 3D printing). Here, additive manufacturing processes bring value in their ability to facilitate rapid prototyping, customization, and optimization of the compression plate and pad bilayer structure. These processes permit the creation of complex geometries and variable density structures that are difficult or even impossible to achieve with traditional plastics manufacturing techniques. Additionally, additive manufacturing offers substantial material flexibility, allowing the use of a variety of polymers and composites suitable for varying degrees of pressure.

Specifically, a 3D printing process can be used based on selective laser sintering (SLS). SLS is a powerful 3D printing technology that uses a laser to sinter powdered polymer material, layer by layer to construct a three-dimensional object based on a digital model. This process excels in the creation of a broad range of geometrical designs and permits the formation of structures with variable densities, offering unparalleled design flexibility. Notably, SLS does not require the use of support structures, unlike some other 3D printing methods, enabling the creation of parts with complex and undercut geometries without subsequent processing. This, in combination with its ability to use a wide range of materials, makes SLS an ideal choice for the fabrication of the T-belt.

### Digital Design, Material Selection, and Printing

The design of the compression backing layer may, for example, be a simple rectangular plate with predetermined dimensions of 4×4×0.25 inches−well within the build volume of an SLS printer (Formlab Fuse 1+build volume: 6.5×6.5×11.8 inches). Powdered polymer resins are readily available in various grades of Nylon™ and polyurethanes that provide flexural modulus (i.e., stiffness) values ranging from 0.75 GPa (semi-rigid) to 10 Gpa (highly rigid). In addition to material selection, the thickness of the plate and incorporation of lattice designs (e.g., rectilinear, honeycomb, triangular, etc.) provide simple parameters to tune the flexural modulus and overall mechanical properties of the compression backing layer. Additionally, lattice design can provide opportunities to lighten the structure and potentially enable the layer to be rolled up into a compact structure without sacrificing the intended mechanical performance.

The second component of the bilayer, e.g., the pressure pad, can be in direct contact with the wounded area of the body and can have different mechanical properties and shapes to ensure appropriate application of force and distribution of pressure to the wound. Materials selection for the pressure pad include SLS polymer powders such as thermoplastic polyurethanes to provide pads with significantly lower flexural modulus and softness. Various cross-sectional profiles, including rectangular, trapezoidal, and hemispherical may be suitable to provide a variety of contact pressure profiles. See FIG. 2 wherein each of elements 120a, 120b, 120c and 120d are separate embodiments of possible cross-sectional profiles.

### REFERENCES

1. Cornelius B, Campbell R, McGauly P. Tourniquets in Trauma Care: A Review of Application, Journal of trauma nursing. 2017;24(3):203-207.
2. Edwards T H, Dubick M A, Palmer L, Pusateri A E. Lessons Learned From the Battlefield and Applicability to Veterinary Medicine—Part 1: Hemorrhage Control.Front Vet Sci. 2021;7.
3. Eastridge B J, Holcomb J B, Shackelford S. Outcomes of traumatic hemorrhagic shock and the epidemiology of preventable death from injury. Transfusion. 2019;59(S2):1423-1428.
4. Mazuchowski E L, Kotwal R S, Janak J C, et al. Mortality review of US Special Operations Command battle-injured fatalities. Journal of Trauma and Acute Care Surgery. 2020;88(5):686-695.
5. Eastridge B J, Mabry R L, Seguin P, et al. Death on the battlefield (2001-2011): Implications for the future of combat casualty care. Journal of Trauma and Acute Care Surgery.2012;73(6):S431-S437.
6. Davis J S, Satahoo S S, Butler F K, et al. An analysis of prehospital deaths: Who can we save? J Trauma Acute Care Surg.2014;77(2):213-218.
7. Kragh Jr J F, Dubick M A. Battlefield tourniquets: lessons learned in moving current care toward best care in an army medical department at war. US Army Medical Department Journal.2016.
8. Flecha I, Naylor J F, Schauer S G, Curtis R A, Cunningham C W. Combat lifesaver-trained, first-responder application of junctional tourniquets: a prospective, randomized, crossover trial. Military Medical Research.2018;5(1):31.
9. Covey D C, Gentchos C E. Field tourniquets in an austere military environment: A prospective case series. Injury.2022;53(10):3240-3247.
10. Van Oostendorp S, Tan E, Geeraedts L. Prehospital control of life-threatening truncal and junctional hemorrhage is the ultimate challenge in optimizing trauma care; a review of treatment options and their applicability in the civilian trauma setting. Scandinavian journal of trauma, resuscitation and emergency medicine.2016;24(1):1-13.
11. Thoolen S J J, Kuypers M I. External Hemorrhage Control Techniques for Human Space Exploration: Lessons from the Battlefield. Wilderness & Environmental Medicine. 2023;34(2):231-242.
12. Arnaud F, Tomori T, Carr W, et al. Exothermic Reaction in Zeolite Hemostatic Dressings: QuikClot ACS and ACS+®.Annals of Biomedical Engineering.2008;36(10):1708-1713.
13. Wang J, Zhang H, Wang J, et al. Efficacy of New Zeolite-Based Hemostatic Gauze in a Gunshot Model of Junctional Femoral Artery Hemorrhage in Swine. Journal of Surgical Research. 2021;263:176-185.
14. Jamal L, Saini A, Quencer K, et al. Emerging approaches to pre-hospital hemorrhage control: a narrative review. Ann Transl Med.2021;9(14):1192.
15. Remondelli M H, Remick K N, Shackelford S A, et al. Casualty Care Implications of Large-Scale Combat Operations. The journal of trauma and acute care surgery.2023.
16. Vrancken S M, Borger van der Burg B L S, DuBose J J, Glaser J J, Hörer T M, Hoencamp R. Advanced Bleeding Control in combat casualty care: an international, expert-based Delphi consensus. The journal of trauma and acute care surgery.2022;93(2):256.
17. Sambor M. Resuscitative Endovascular Balloon Occlusion of the Aorta for Hemorrhage Control in Trauma Patients: An Evidence-Based Review.J Trauma Nurs.2018;25(1):33-37.
18. Tovmassian R V, Kragh J F, Jr., Dubick M A, Billings S, Blackbourne L H. Combat ready clamp medic technique. J Spec Oper Med.2012;12(4):72-78.
19. Meusnier J-G, Dewar C, Mavrovi E, Caremil F, Wey P-F, Martinez J-Y. Evaluation of two junctional tourniquets used on the battlefield. J Special Operations Med. 2016;16(3):41-46. Li Y, Li H, Xiao L, et al. Hemostatic Efficiency and Wound Healing Properties of Natural Zeolite Granules in a Lethal Rabbit Model of Complex Groin Injury. Materials(Basel). 2012;5(12):2586-2596. doi:2510.3390/ma5122586.eCollection 5122012 Dec.

### FIGURE REFERENCE NUMBERS

1—trauma belt
10—end surface of top arm
20—interior surface of top arm
30—interior surface of stem
40—stem
50—interior surface of bottom arm
60—end surface of bottom arm
70—top arm
80—bottom arm
100—pad
110—compression plate
120a—pressure pad block type
120b—pressure pad with curved edges
120c—pressure pad with trapezoidal shape
120d—pressure pad with hemispherical shape
200—stretchable trauma belt
210—stretchable belt
220—hook-and-loop fastener coupler
230—pad
240—compression plate
250—pressure pad
250a—pressure pad block type
250b—pressure pad with curved edges
250c—pressure pad with trapezoidal shape
250d—pressure pad with hemispherical shape

1. A trauma belt (1) for non-tourniquetable hemorrhage control of a patient having an entry wound in a shoulder bundle, groin, femoral, abdominal, back, or chest region, said trauma belt (1) comprising a rigid top arm (70), a rigid bottom arm (80) and a stem (40) connecting the top arm (70) and the bottom arm (80),
a pad (100) connected to the interior surface (20) of the top arm (70),
wherein the stem (40) is configured to provide constricting force between the top arm (70) and bottom arm (80) to provide pressure of the pad (100) against the entry wound.

2. The trauma belt (1) of claim 1, wherein a second pad (100) is connected to an interior surface (50) of the bottom arm (80) to press against an exit wound.

3. The trauma belt (1) of claim 1, wherein the pad (100) is detachable from the trauma belt (1).

4. The trauma belt (1) of claim 1, wherein the pad (100) has a compression plate (110) and a pressure pad (120).

5. The trauma belt (1) of claim 4, wherein the pressure pad (120) is a block type (120a), or has curved edges (120b), or is trapezoidal shaped (120c) or is hemispherical shaped (120d).

6. The trauma belt (1) of claim 1, wherein the trauma belt (1) is formed using additive manufacturing.

7. The trauma belt (1) of claim 6, wherein an exterior wall of the trauma belt (1) has a thickness of from about 10 mm to about 30 mm.

8. The trauma belt (1) of claim 6, wherein the trauma belt (1) comprises an internal infill percentage of from about 10% to about 100%, based on the total volume of the trauma belt (1).

9. The trauma belt (1) of claim 1, wherein the trauma belt (1) is configured to have sufficient tension to ensure that the pad (100) provides 20-700 mm Hg pressure inward towards the wound.

10. The trauma belt (1) of claim 1, wherein the thickness of the compression plate (110) is 0.1-1 inch.

11. The trauma belt (1) of claim 1, wherein an area of the pad (100) in the horizontal plane is 4-36 square inches.

12. The trauma belt (1) of claim 1, wherein the pad (100) further comprises one or more components selected from the group consisting of a quick clot, an anticoagulant, an anesthetic, and an antimicrobial.

13. The trauma belt (1) of claim 1, wherein a distance between a lower surface of the pad (100) attached to the top arm (70) and an upper surface of the bottom arm (80) is from about 6 inches to about 24 inches.

14. A stretchable trauma belt (200) for non-tourniquetable hemorrhage control of a patient having an entry wound in a shoulder bundle, groin, femoral, abdominal, back, or chest region, said trauma belt (200) comprising
a stretchable belt (210),
a pad (230) adhered to the stretchable belt (210),
a fastener (220) configured to fasten two or more opposite edges of the stretchable belt (200) to form a closed loop to provide a constricting force and pressure of the pad (230) against the entry wound.

15. The stretchable trauma belt (200) of claim 14, comprising a plurality of pads (230).

16. The stretchable trauma belt (200) of claim 14, wherein the pad (230) is detachable from the stretchable belt (210).

17. The stretchable trauma belt (200) of claim 14, wherein the pad (230) comprises a compression plate (240) and a pressure pad (250), wherein the compression plate has a top side adhered to the pressure pad and a bottom side adhered to the stretchable belt.

18. The stretchable trauma belt (200) of claim 17, wherein the compression plate (240) is rigid or semi-rigid.

19. The stretchable trauma belt (200) of claim 15, wherein each of the plurality of pads (230) are arranged side by side along the stretchable belt such that their longitudinal edges are adjacent to each other.

20. The stretchable trauma belt (200) of claim 19, wherein each of the plurality of pads (230) comprises an elongate, rigid compression plate (240) and a pressure pad (250).

21. The stretchable trauma belt (200) of claim 20, wherein the stretchable belt (210) is made of a material selected from the group consisting of high-strength textile, elastomeric sheet, and polymer film.
